(19) United States
(12) Patent Application Publication
(10) Pub. No.: US 2016/0141517 A1
(43) Pub. Date: May 19, 2016

(54) COMPOUND AND METHODS FOR PREPARING THE SAME AND ITS APPLICATIONS

(71) Applicant: EverDisplay Optonics (Shanghai) Limited, Shanghai (CN)

(72) Inventor: Hongling YANG, Shanghai (CN)

(73) Assignee: EverDisplay Optonics (Shanghai) Limited, Shanghai (CN)

(21) Appl. No.: 14/939,354

(22) Filed: Nov. 12, 2015

(30) Foreign Application Priority Data
Nov. 13, 2014 (CN) 201410640789.7

(51) Int. Cl. H01L 51/00; C09K 11/02; C07D 403/10

(52) U.S. Cl. CPC H01L 51/0072; C07D 403/10; H01L 51/0067; C09K 11/025; H01L 51/0071; H01L 51/5012

(57) ABSTRACT

The present invention provides a compound having the following formula (I):

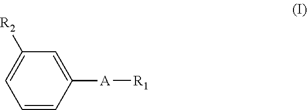

R₂ is an electron withdrawing group; A represents a structure with 0 to 20 benzene rings connected in sequence, R₁ is an electron donating group or to form a cyclic electron donating group with parts of C atoms of the last benzene ring. The present invention also provides an organic layer of OLED devices. The present invention also provides a method for synthesizing the compound represented by formula (I). The OLED material with the novel structure provided by the present invention can be applied to an electron-transporting layer, a light emitting layer, a hole transporting layer. Hence, the display devices consisting of the aforesaid OLED material have the advantages of swift response, low power consumption, and wide viewing angle.

### CROSS-REFERENCE TO RELATED APPLICATIONS

The present application claims priority to and the benefit of Chinese Patent Application No. CN 201410640789.7, filed on Nov. 3, 2014, the entire content of which is incorporated herein by reference.

### BACKGROUND OF THE INVENTION

1. Field of the Invention

The present invention relates to a compound, more specifically, relates to a compound used for OLED host material, and the method for preparing the same, as well as applications of the same.

2. Description of the Related Art

Currently, displays mainly consist of TFTs (Thin Film Transistor, thin-film transistor)-LCD. Since the TFT-LCD is non-self-luminous display which emits light through backlight. The light generated from the backlight passes in sequence through the polarizing sheet, glass substrate, liquid crystal layer, color filters, and other related components in the TFT-LCD panel to achieve the final arrival at people's sight and for imaging, which realizes the function of the display.

LED display integrates microelectronic technology, computer technology, information processing and has become the most advantageous display media used in the public due to its beautiful color, wide dynamic range, high brightness, long service life as well as the advantages of stable and reliable and so on. Hence, LED display has been widely used in large squares, commercial advertisements, sports venues, Stock Exchanges to meet the demand of different environments.

OLED display is the next generation flat panel display which is similar to and better than the LCD. OLED has a very simple sandwich structure, i.e., having a very thin layer of organic materials between two layers of electrodes. When any current is passed through, these organic materials will emit light. Compared with LCD display, OLED has many advantages: as the OLED emits light itself with no backlight, OLED display can be designed and manufactured to be thinner and lighter with a larger viewing angle, beautiful color and significantly low energy consumption. Due to the aforesaid advantages, OLED has been widely used in MP3, mobile phones and other mobile electronic devices, and has been gradually applied to PC monitors, laptops, televisions and other large-size display areas.

The basic structure of an OLED is formed by a thin and transparent indium tin oxide (ITO), with a characteristic of semi conductive, connected to the positive electrode, and a metal cathode, by which a sandwich structure is formed, such an OLED as disclosed in U.S. Pat. No. 4,769,292. The entire structure layers include: a hole transport layer (HTL), an emitting layer (EL) and an electron transport layer (ETL). When the a suitable voltage is supplied to the device, the positive holes and the cathode charge will be combined in the light emitting layer to emit lights which comprises RGB primary lights, i.e., red light, green light and blue light, generated based on different formulations, to constitute the basic colors. One of the properties of the OLED is light-emitting by itself with no backlight, which is different from the TFT LCD. Hence, visibility and brightness of the OLED are higher, and the demand for voltage is low while the electrical efficiency is higher. Moreover, the OLED also possess the advantages of swift in response, light in weight, thin in thickness and simple in structure, and low in cost.

### SUMMARY OF THE INVENTION

An aspect of an embodiment of the present disclosure is directed toward a compound applied to an electron-transporting layer, a light emitting layer, a hole transporting layer of an OLED having the advantages of swift response, low power consumption, and wide viewing angle.

Another aspect of an embodiment of the present disclosure is directed toward an OLED device employing the aforesaid compound.

Another aspect of an embodiment of the present disclosure is directed toward a method for preparing the aforesaid compound.

An embodiment of the present disclosure provides a compound, having the following formula (I):

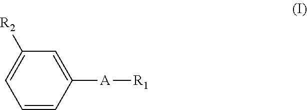

wherein, R₂ is an electron withdrawing group; A represents a structure with 0 to 20 benzene rings connected in sequence; R₁ is an electron donating group or to form a cyclic electron donating group with parts of carbon atoms of the last benzene ring.

According to one embodiment of the present disclosure, wherein the A at least has a structure represented by the following formula:

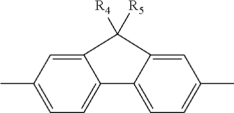

wherein, R₄ and R₅ is independently selected from a group consisting of H, C1 to C5 alkyl, phenyl, and benzyl.

According to one embodiment of the present disclosure, wherein the electron donating group R₁ is:

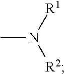

wherein, R¹ and R² are independently selected from a group consisting of H, C1 to C5 alkyl, C1 to C2 alkyl replaced by aryl, and aryl, or R¹ and R² together with N forms a nitrogen heterocycle; wherein, the aryl is contained in at least one of the R¹ and R²;

wherein, the aryl is contained in at least one of the R¹ and R².

According to one embodiment of the present disclosure, wherein R¹ and R² is selected from a group consisting of H, C1 to C5 alkyl and

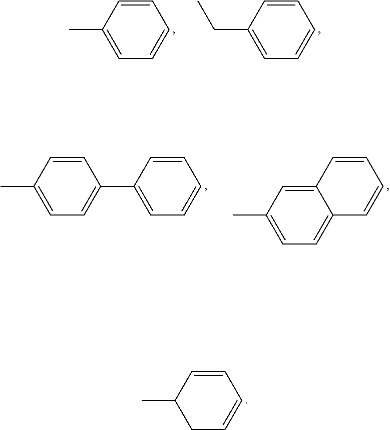

According to one embodiment of the present disclosure, wherein the nitrogen heterocycle formed by R¹ and R² together with N is selected from

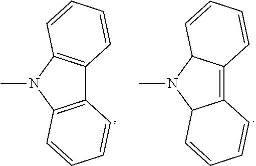

According to one embodiment of the present disclosure, wherein the cyclic electron donating group formed by R1 with parts of carbon atoms of the last benzene ring is selected from

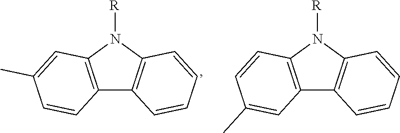

wherein, R is selected from a group consisting of H, C1 to C5 alkyl, and aryl.

According to one embodiment of the present disclosure, wherein the R₂ is selected from

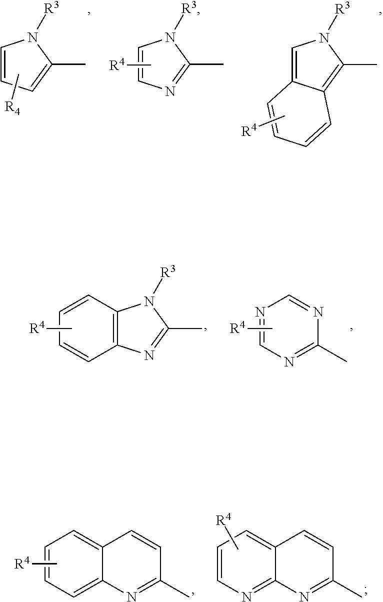

wherein, R³ and R⁴ is independently selected from a group consisting of H, C1 to C5 alkyl, C1 to C2 alkyl replaced by aryl, and aryl.

According to one embodiment of the present disclosure, wherein the compound has the following formula (II):

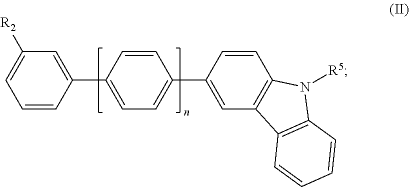

wherein, n is an integer from 0 to 20, R⁵ is selected from a group consisting of H, C1 to C5 alkyl, C1 to C2 alkyl replaced by aryl, and aryl.

According to one embodiment of the present disclosure, wherein the compound has the following formula (III):

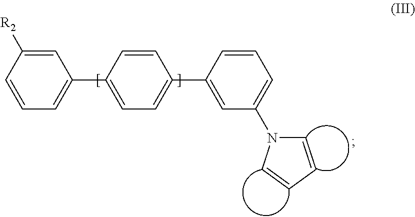

wherein, n is an integer from 0 to 20, N heterocyclic is a polycyclic electron donating group.

Another embodiment of the present disclosure provides an OLED device, comprising: a cathode; an anode; and an organic layer located between the cathode and the anode; wherein, the organic layer comprises the compound represented by formula (I):

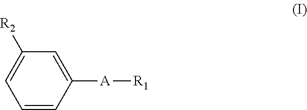

wherein, R, is an electron withdrawing group; A represents a structure with 0 to 20 benzene rings connected in sequence; R₁ is an electron donating group or to form a cyclic electron donating group with parts of carbon atoms of the last benzene ring.

Another embodiment of the present disclosure provides a method for synthesizing the compound represented by formula (I), wherein the compound is synthesized by Suzuki reaction synthesis;

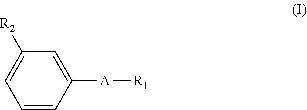

wherein, R₂ is an electron withdrawing group; A represents a structure with 0 to 20 benzene rings connected in sequence, R₁ is an electron donating group or to form a cyclic electron donating group with parts of carbon atoms of the last benzene ring.

### DETAILED DESCRIPTION

The present invention will now be described more fully hereinafter, in which exemplary embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” or “includes” and/or “including” or “has” and/or “having” when used herein, specify the presence of stated features, regions, integers, steps, operations, elements, and/or components, but do not preclude the presence or addition of one or more other features, regions, integers, steps, operations, elements, components, and/or groups thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

As used herein, “around”, “about” or “approximately” shall generally mean within 20 percent, preferably within 10 percent, and more preferably within 5 percent of a given value or range. Numerical quantities given herein are approximate, meaning that the term “around”, “about” or “approximately” can be inferred if not expressly stated.

As used herein, the term “plurality” means a number greater than one.

The present invention provides a compound which can be applied to an electron-transporting layer, a light emitting layer, a hole transporting layer of the OLED material. The compound has the following formulas:

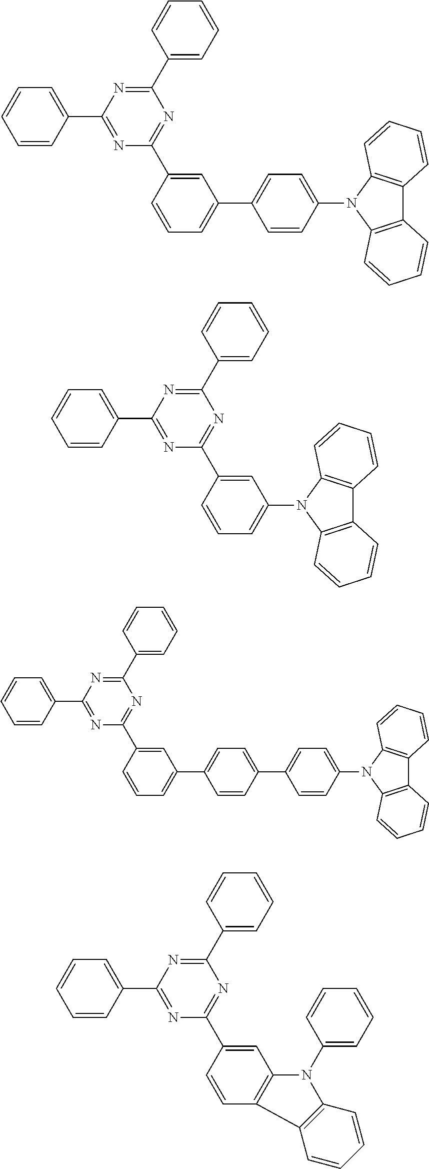 [SMILES: c1ccc(-c2nc(-c3ccccc3)nc(-c3cccc(-c4ccc(-n5c6ccccc6c6ccccc65)cc4)c3)n2)cc1 ; c1ccc(-c2nc(-c3ccccc3)nc(-c3cccc(-n4c5ccccc5c5ccccc54)c3)n2)cc1 ; c1ccc(-c2nc(-c3ccccc3)nc(-c3cccc(-c4ccc(-c5ccc(-n6c7ccccc7c7ccccc76)cc5)cc4)c3)n2)cc1 ; c1ccc(-c2nc(-c3ccccc3)nc(-c3ccc4c5ccccc5n(-c5ccccc5)c4c3)n2)cc1]

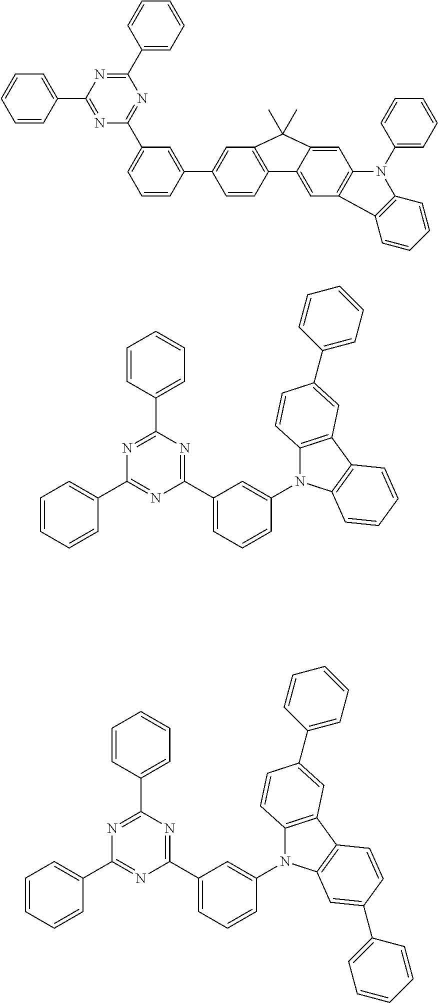 [SMILES: CC1(C)c2cc(-c3cccc(-c4nc(-c5ccccc5)nc(-c5ccccc5)n4)c3)ccc2-c2cc3c4ccccc4n(-c4ccccc4)c3cc21 ; c1ccc(-c2ccc3c(c2)c2ccccc2n3-c2cccc(-c3nc(-c4ccccc4)nc(-c4ccccc4)n3)c2)cc1 ; c1ccc(-c2ccc3c(c2)c2ccc(-c4ccccc4)cc2n3-c2cccc(-c3nc(-c4ccccc4)nc(-c4ccccc4)n3)c2)cc1]

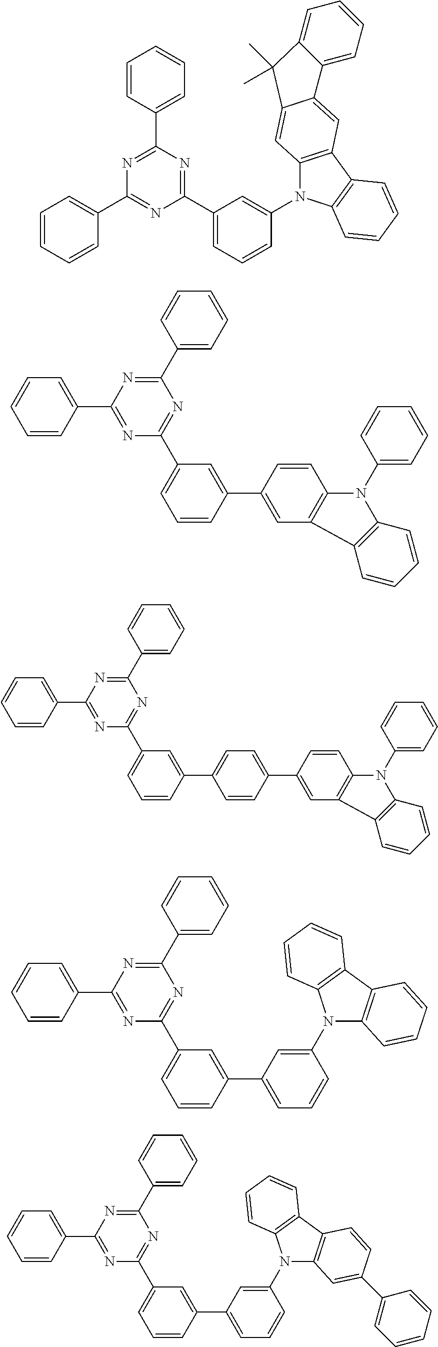

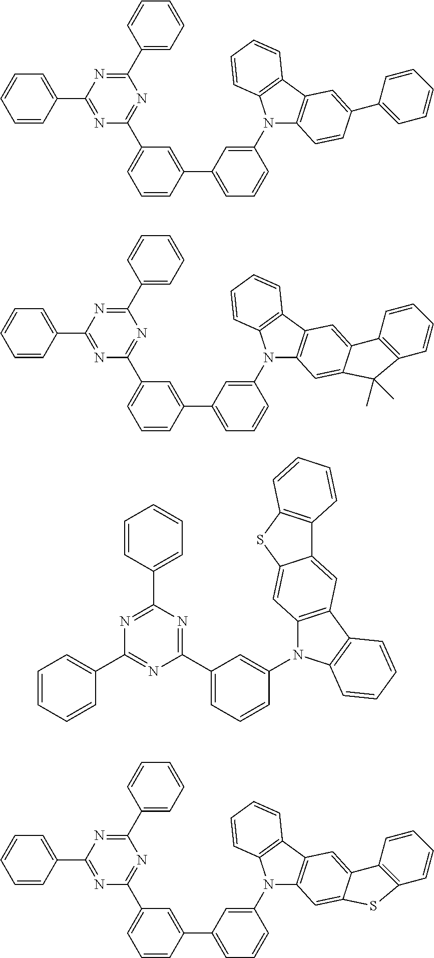 [SMILES: c1ccc(-c2ccc3c(c2)c2ccccc2n3-c2cccc(-c3cccc(-c4nc(-c5ccccc5)nc(-c5ccccc5)n4)c3)c2)cc1 ; CC1(C)c2ccccc2-c2cc3c4ccccc4n(-c4cccc(-c5cccc(-c6nc(-c7ccccc7)nc(-c7ccccc7)n6)c5)c4)c3cc21 ; c1ccc(-c2nc(-c3ccccc3)nc(-c3cccc(-n4c5ccccc5c5cc6c(cc54)sc4ccccc46)c3)n2)cc1 ; c1ccc(-c2nc(-c3ccccc3)nc(-c3cccc(-c4cccc(-n5c6ccccc6c6cc7c(cc65)sc5ccccc57)c4)c3)n2)cc1]

### PREPARATION EXAMPLE 1

Compound A is prepared by the following method:

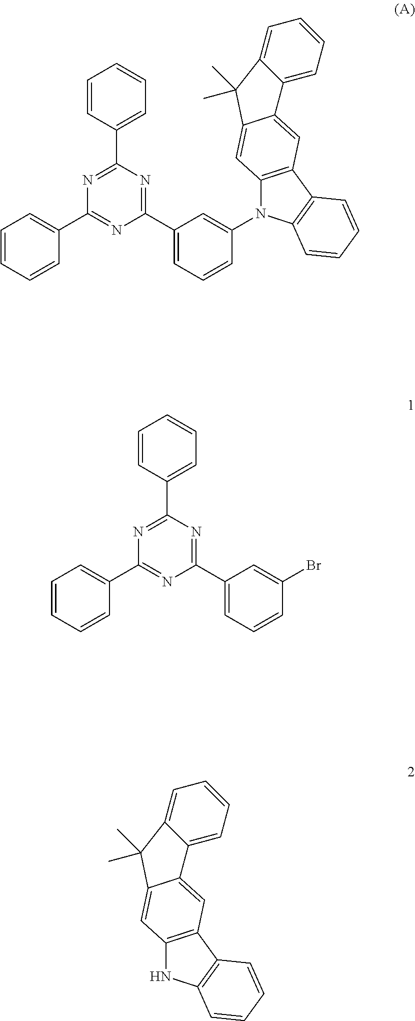 [SMILES: CC1(C)c2ccccc2-c2cc3c4ccccc4n(-c4cccc(-c5nc(-c6ccccc6)nc(-c6ccccc6)n5)c4)c3cc21 ; Brc1cccc(-c2nc(-c3ccccc3)nc(-c3ccccc3)n2)c1 ; CC1(C)c2ccccc2-c2cc3c(cc21)[nH]c1ccccc13]

Introduce a mixture comprising 0.1 mol of Intermediate 1, 0.1 mol of Intermediate 2, potassium tert-butoxide, palladium acetate tri-tert-butylphosphine tetrafluoroborate and toluene (1000 ml) into a reaction container. Heat and reflux the aforesaid mixture for 24 hours with the protection of nitrogen gas, and then, cooling and removing the toluene. Dichloromethane is added into the reaction container, and then a washing process with water and a drying process are performed, the crude product was passed through the column, and then performing a recrystallization process and a purifying process with dichloromethane and ethanol to obtain the Compound A.

Characterization of the molecular weight of the obtained compound A is: MS 590.24;

H-NMR: 1.67(6H), 7.22(2H), 7.32(4H), 7.48(4H), 7.50(2H), 7.3(1H), 7.21(1H), 7.43(2H), 7.0(2H), 7.33(1H), 8.06(1H), 7.61(2H), 7.36(2H).

### PREPARATION EXAMPLE 2

Compound B is prepared by the following method:

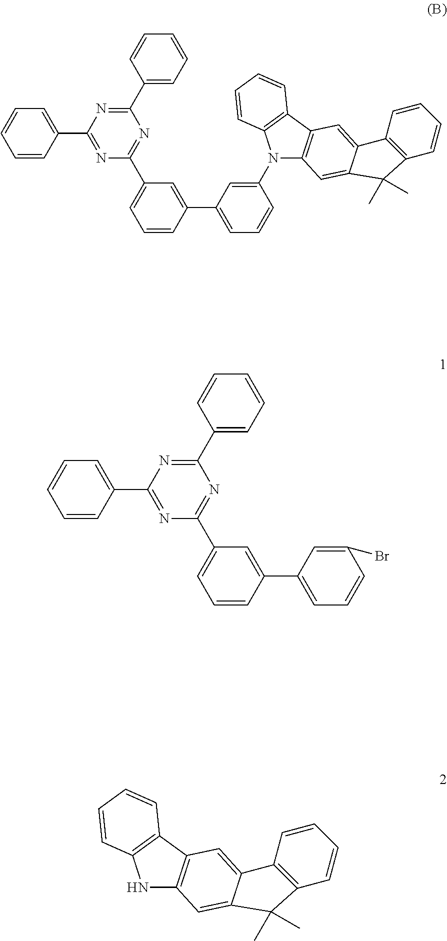 [SMILES: CC1(C)c2ccccc2-c2cc3c4ccccc4n(-c4cccc(-c5cccc(-c6nc(-c7ccccc7)nc(-c7ccccc7)n6)c5)c4)c3cc21 ; Brc1cccc(-c2cccc(-c3nc(-c4ccccc4)nc(-c4ccccc4)n3)c2)c1 ; CC1(C)c2ccccc2-c2cc3c(cc21)[nH]c1ccccc13]

The preparation for the compound B is the same as described in the preparation example 1.

Characterization of the molecular weight of the obtained compound B is: MS 666.27;

H-NMR: 1.67(6H), 7.22(2H), 7.32(4H), 7.48(4H), 7.50(2H), 7.3(2H), 7.21(2H), 7.43(2H), 7.0(2H), 7.33(2H), 8.06(1H), 7.61(3H), 7.36(2H).

### PREPARATION EXAMPLE 3

Compound C is prepared by the following method:

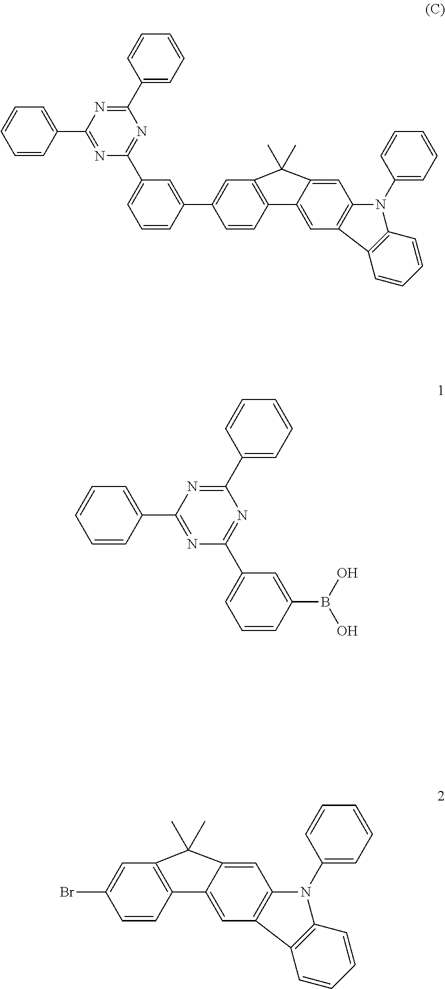 [SMILES: CC1(C)c2cc(-c3cccc(-c4nc(-c5ccccc5)nc(-c5ccccc5)n4)c3)ccc2-c2cc3c4ccccc4n(-c4ccccc4)c3cc21 ; OB(O)c1cccc(-c2nc(-c3ccccc3)nc(-c3ccccc3)n2)c1 ; CC1(C)c2cc(Br)ccc2-c2cc3c4ccccc4n(-c4ccccc4)c3cc21]

The preparation for the compound C is the same as described in the preparation example 1.

Characterization of the molecular weight of the obtained compound C is: MS 666.27;

H-NMR: 1.67(6H), 7.22(2H), 7.32(4H), 7.48(4H), 7.50(2H), 7.3(2H), 7.21(2H), 7.43(2H), 7.0(2H), 7.33(2H), 8.06(1H), 7.61(3H), 7.36(2H).

### PREPARATION EXAMPLE 4

Compound D is prepared by the following method:

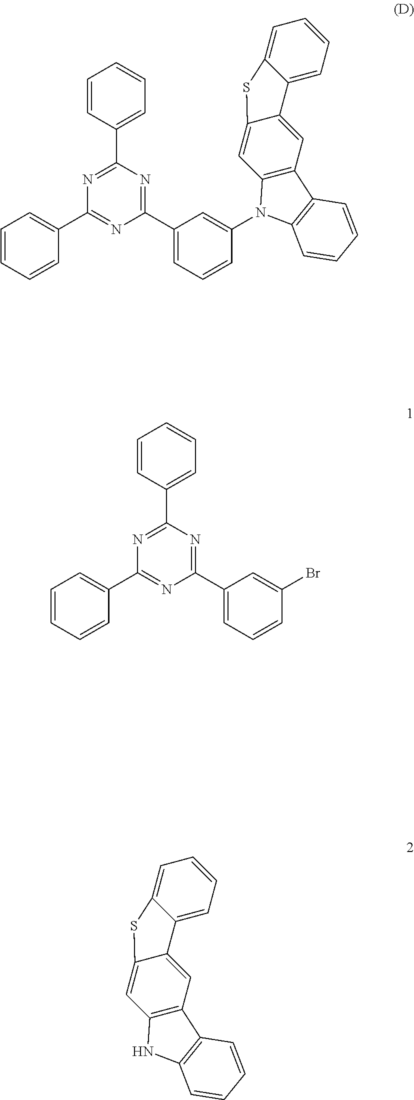 [SMILES: c1ccc(-c2nc(-c3ccccc3)nc(-c3cccc(-n4c5ccccc5c5cc6c(cc54)sc4ccccc46)c3)n2)cc1 ; Brc1cccc(-c2nc(-c3ccccc3)nc(-c3ccccc3)n2)c1 ; c1ccc2c(c1)[nH]c1cc3sc4ccccc4c3cc12]

The preparation for the compound D was the same as described in the preparation example 1.

Characterization of the molecular weight of the obtained compound D is: MS 580.69;

H-NMR: 7.22(2H), 7.32(4H), 7.48(4H), 7.50(2H), 7.3(1H), 7.21(1H), 7.43(2H), 7.0(2H), 7.33(1H), 8.06(1H), 7.61(2H), 7.36(2H).

### PREPARATION EXAMPLE 5

Compound E is prepared by the following method:

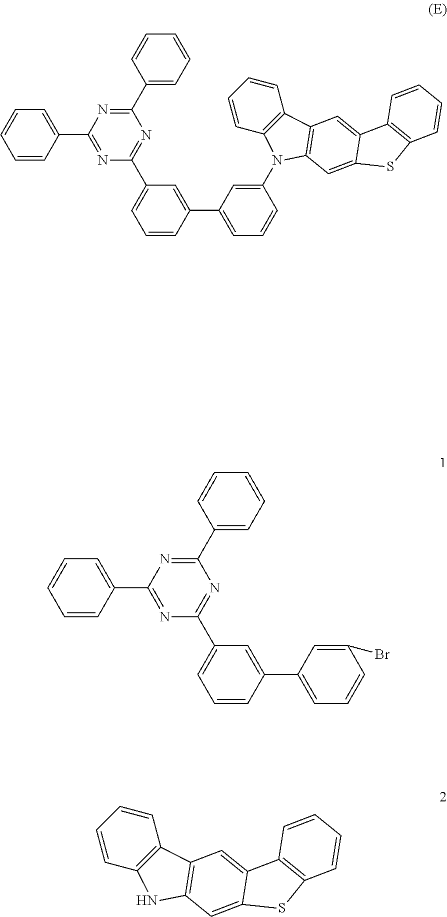 [SMILES: c1ccc(-c2nc(-c3ccccc3)nc(-c3cccc(-c4cccc(-n5c6ccccc6c6cc7c(cc65)sc5ccccc57)c4)c3)n2)cc1 ; Brc1cccc(-c2cccc(-c3nc(-c4ccccc4)nc(-c4ccccc4)n3)c2)c1 ; c1ccc2c(c1)[nH]c1cc3sc4ccccc4c3cc12]

The preparation for the compound E is the same as described in the preparation example 1.

Characterization of the molecular weight of the obtained compound E is: MS 656.79;

H-NMR: 7.22(2H), 7.32(4H), 7.48(4H), 7.50(2H), 7.3(2H), 7.21(2H), 7.43(2H), 7.0(2H), 7.33(2H), 8.06(1H), 7.61(3H), 7.36(2H).

### PREPARATION EXAMPLE 6

Compound F is prepared by the following method:

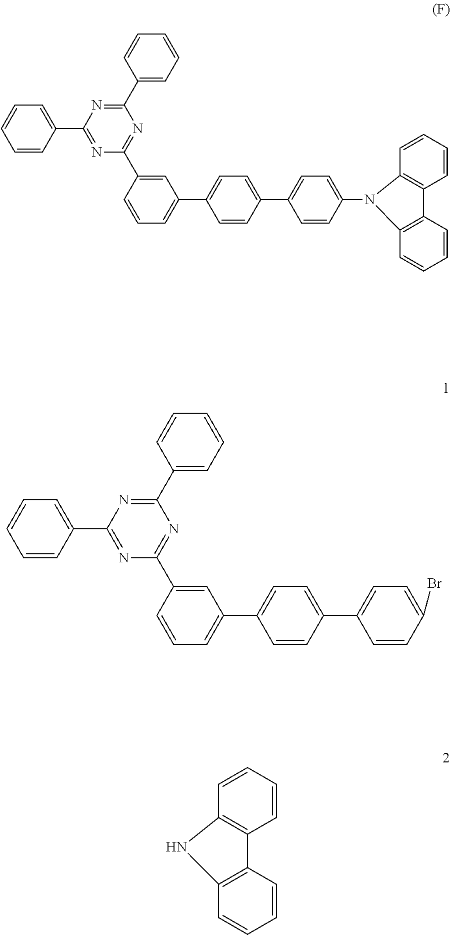 [SMILES: c1ccc(-c2nc(-c3ccccc3)nc(-c3cccc(-c4ccc(-c5ccc(-n6c7ccccc7c7ccccc76)cc5)cc4)c3)n2)cc1 ; Brc1ccc(-c2ccc(-c3cccc(-c4nc(-c5ccccc5)nc(-c5ccccc5)n4)c3)cc2)cc1 ; c1ccc2c(c1)[nH]c1ccccc12]

The preparation for the compound F is the same as described in the preparation example 1.

Characterization of the molecular weight of the obtained compound F is: MS 626.74;

H-NMR: 7.22(2H), 7.32(4H), 7.48(4H), 7.50(2H), 7.3(2H), 7.21(2H), 7.43(2H), 7.0(2H), 7.33(2H), 8.06(1 H), 7.61(3H), 7.36(2H).

The manufacture of the device:

### Embodiment 1

The transparent anode electrode ITO substrate is ultrasonically cleaned in isopropanol for 5-10 minutes, and then is exposed to UV light for 20-30 minutes, then is treated by plasma for 5-10 minutes. The treated ITO substrate is then put into an evaporation apparatus. Firstly, it was in sequence deposited with an NPB layer of 30-50 nm, a compound A, an Ir(ppy)3 of 5-10%, an Alq3 (8-hydroxyquinoline aluminum) layer of 20-40 nm, and LiF layer of 0.5-2 nm, and metal Al layer of 100-200 nm.

### Embodiment 2

The compound A in Embodiment 1 is replaced by Compound B.

### Embodiment 3

The compound A in Embodiment 1 is replaced by Compound C.

### Embodiment 4

The compound A in Embodiment 1 is replaced by Compound D.

### Embodiment 5

The compound A in Embodiment 1 is replaced by Compound E.

### Embodiment 6

The compound A in Embodiment 1 is replaced by Compound F.

### Embodiment 7

The compound A in Embodiment 1 is replaced by CBP, and Alp3 in Embodiment 1 is replaced by Compound A.

### Embodiment 8

The compound A in Embodiment 1 is replaced by CBP, and Alp3 in Embodiment 1 is replaced by Compound B.

### Embodiment 9

The compound A in Embodiment 1 is replaced by CBP, and Alp3 in Embodiment 1 is replaced by Compound C.

### Embodiment 10

The compound A in Embodiment 1 is replaced by CBP, and Alp3 in Embodiment 1 is replaced by Compound D.

### Embodiment 11

The compound A in Embodiment 1 is replaced by CBP, and Alp3 in Embodiment 1 is replaced by Compound E.

### Embodiment 12

The compound A in Embodiment 1 is replaced by Compound CBP, and Alp3 in Embodiment 1 is replaced by Compound F.

### Comparative Embodiment

The compound A in Example 1 is replaced by CBP.

Wherein,

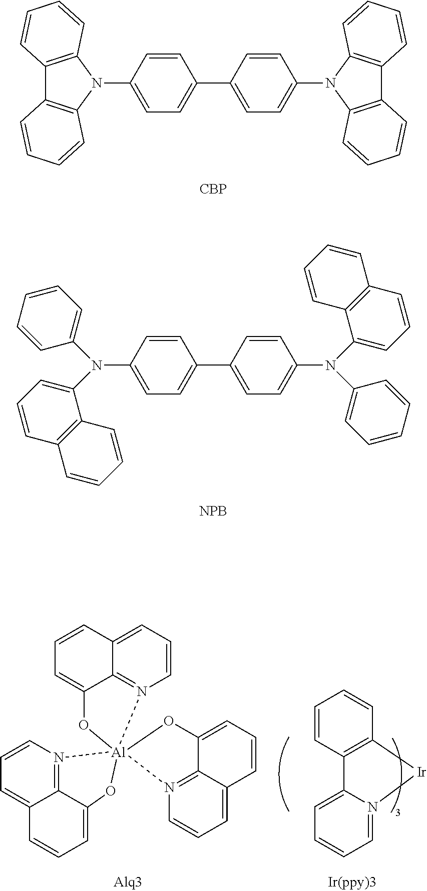

The materials of an OLED are as follows:

Embodiment 1: ITO/NPB/Compound A: Ir(ppy)3/Alq3/LiF/Al;

Embodiment 2: ITO/NPB/Compound B: Ir(ppy)3/Alq3/LiF/Al;

Embodiment 3: ITO/NPB/Compound C: Ir(ppy)3/Alq3/LiF/Al;

Embodiment 4: ITO/NPB/Compound D: Ir(ppy)3/Alq3/LiF/Al;

Embodiment 5: ITO/NPB/Compound E: Ir(ppy)3/Alq3/LiF/Al;

Embodiment 6: ITO/NPB/Compound F: Ir(ppy)3/Alq3/LiF/Al;

Embodiment 7: ITO/NPB/CBP: Ir(ppy)3/Compound A/LiF/Al;

Embodiment 8: ITO/NPB/CBP: Ir(ppy)3/Compound B/LiF/Al;

Embodiment 9: ITO/NPB/CBP: Ir(ppy)3/Compound C/LiF/Al;

Embodiment 10: ITO/NPB/CBP: Ir(ppy)3/Compound D/LiF/Al;

Embodiment 11: ITO/NPB/CBP: Ir(ppy)3/Compound E/LiF/Al;

Embodiment 12: ITO/NPB/CBP: Ir(ppy)3/Compound F/LiF/Al;

Comparative embodiment: ITO/NPB/CBP: Ir(ppy)3/Alq3/LiF/Al.

The test results of OLED devices under the test conditions lower than 1000 nits are shown in Table 1 below.

TABLE 1
the test results of the OLED devices

| Device | Cd/A | Driver Voltage | CIEx | CIEy |
|---|---|---|---|---|
| Comparative | 10 cd/A | 4.6 V | 0.33 | 0.64 |
| example | | | | |
| Embodiment 1 | 20 cd/A | 4.5 V | 0.33 | 0.64 |
| Embodiment 2 | 15 cd/A | 4.4 V | 0.33 | 0.64 |
| Embodiment 3 | 19 cd/A | 4.3 V | 0.33 | 0.64 |
| Embodiment 4 | 15 cd/A | 4.5 V | 0.33 | 0.64 |
| Embodiment 5 | 12 cd/A | 4.7 V | 0.33 | 0.64 |
| Embodiment 6 | 23 cd/A | 4.8 V | 0.33 | 0.64 |
| Embodiment 7 | 21 cd/A | 4.9 V | 0.33 | 0.64 |
| Embodiment 8 | 14 cd/A | 4.8 V | 0.33 | 0.64 |
| Embodiment 9 | 22 cd/A | 5.0 V | 0.33 | 0.64 |
| Embodiment 10 | 17 cd/A | 4.7 V | 0.33 | 0.64 |
| Embodiment 11 | 15 cd/A | 4.6 V | 0.33 | 0.64 |
| Embodiment 12 | 21 cd/A | 4.5 V | 0.33 | 0.64 |

The above specific embodiments of the present invention have been described in detail, but only as an example, the present invention is not limited to the specific embodiments described above. The skilled in the art, any equivalent modifications and substitutions of the present invention are also in the scope of the invention. Therefore, equalization changes and modifications without departing from the spirit and scope of the present invention made should fall within the scope of the present invention.

What is claimed is:

1. A compound, having the following formula (I): 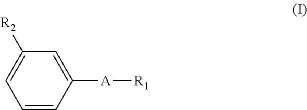
wherein, R₂ is an electron withdrawing group; A represents a structure with 0 to 20 benzene rings connected in sequence; R₁ is an electron donating group or to form a cyclic electron donating group with parts of carbon atoms of the last benzene ring.

2. The compound according to claim 1, wherein the A at least has a structure represented by the following formula: 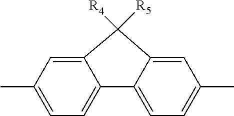
wherein, R₄ and R₅ is independently selected from a group consisting of H, C1 to C5 alkyl, phenyl and benzyl.

3. The compound according to claim 1, wherein the electron donating group R₁ is: 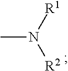
wherein, R¹ and R² are independently selected from a group consisting of H, C1 to C5 alkyl, C1 to C2 alkyl replaced by aryl, and aryl, or R¹ and R² together with N form a nitrogen heterocycle; wherein, the aryl is contained in at least one of the R¹ and R².

4. The compound according to claim 3, wherein R¹ and R² is independently selected from a group consisting of H, C1 to C5 alkyl and

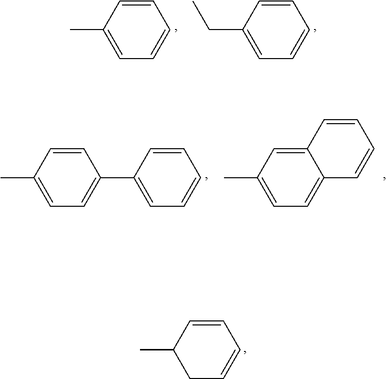

5. The compound according to claim 3, wherein the nitrogen heterocycle formed by R¹ and R² together with N is selected from

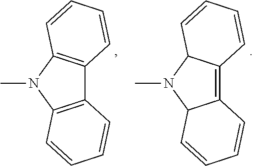

6. The compound according to claim 1, wherein the cyclic electron donating group formed by R1 with parts of carbon atoms of the last benzene ring is selected from 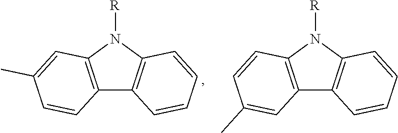
wherein, R is selected from a group consisting of H, C1 to C5 alkyl, and aryl.

7. The compound according to claim 1, wherein the R₂ is selected from 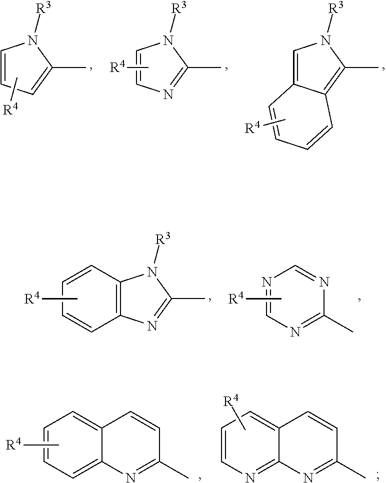
wherein, R³ and R⁴ is independently selected from a group consisting of H, C1 to C5 alkyl, C1 to C2 alkyl replaced by aryl, and aryl.

8. The compound according to claim 1, wherein the compound has the following formula (II): 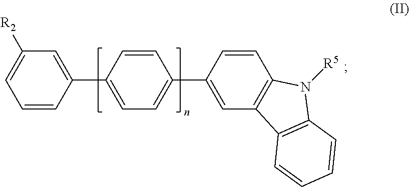
wherein, n is an integer from 0 to 20, R⁵ is selected from a group consisting of H, C1 to C5 alkyl, C1 to C2 alkyl replaced by aryl, and aryl.

9. The compound according to claim 1, wherein the compound has the following formula (III): 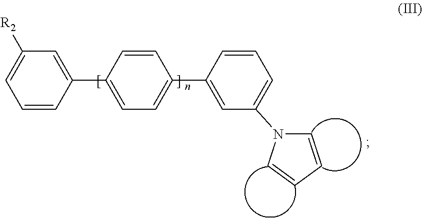
wherein, n is an integer from 0 to 20, N heterocyclic is a polycyclic electron donating group.

10. An OLED device, comprising:
a cathode;
an anode; and
an organic layer located between the cathode and the anode;
wherein, the organic layer comprises the compound represented by formula (I): 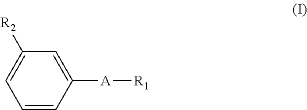
wherein, R₂ is an electron withdrawing group; A represents a structure with 0 to 20 benzene rings connected in sequence; R₁ is an electron donating group or to form a cyclic electron donating group with parts of carbon atoms of the last benzene ring.

11. A method for synthesizing the compound represented by formula (I), wherein the compound is synthesized by Suzuki reaction synthesis; 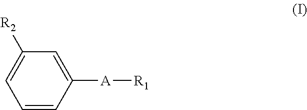
wherein, R₂ is an electron withdrawing group; A represents a structure with 0 to 20 benzene rings connected in sequence, R₁ is an electron donating group or to form a cyclic electron donating group with parts of carbon atoms of the last benzene ring.
